(19) United States
(12) Patent Application Publication
(10) Pub. No.: US 2006/0258717 A1
(43) Pub. Date: Nov. 16, 2006

(54) ORGANIC COMPOUNDS

(76) Inventors: Anna Ottlecz, Basel (CH); Rudolf Karl Andreas Giger, Muttenz (CH); Carlos Alvarez, Riehen (CH)

Correspondence Address: HOFFMAN WARNICK & D'ALESSANDRO, LLC, 75 STATE STREET, 14TH FLOOR, ALBANY, NY, 12207, US

(21) Appl. No.: 11/460,355

(22) Filed: Jul. 27, 2006

Related U.S. Application Data
(63) Continuation of application No. PCT/US05/03427, filed on Jan. 28, 2005.

(30) Foreign Application Priority Data
Jan. 30, 2004 (GB) GB 0402100.2

(51) Int. Cl. A61K 31/4439; C07D 403/02

(52) U.S. Cl. 514/341; 546/272.7

(57) ABSTRACT

The present invention provides methods and uses of imidazolylalkyl-pyridines in the treatment of ocular disorders. A first aspect of the invention provides a method of treating ocular neovascularization comprising administering to a subject in need of such treatment a therapeutically effective amount of a compound having the formula (I) in free base or physiologically acceptable acid addition salt form, 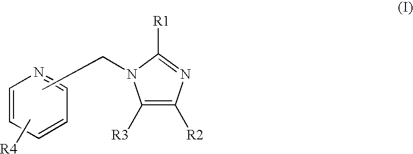
wherein R₁. is hydrogen, lower alkyl, halogen with an atomic number of 9 to 35 or amino optionally mono- or disubstituted by lower alkyl, R₂. and R₃. independently of one another are hydrogen or lower alkyl, R₄. is hydrogen, hydroxy, lower alkyl, lower alkoxy or halogen with an atomic number of 9 to 35, and the bridge between the pyridine and the imidazole, illustrated as methylene, is methylene or ethylene.

### CLAIM FOR PRIORITY UNDER 35 USC §120

This is a continuation of International Application PCT/US2005/003427, with an International Filing Date of Jan. 28, 2005, currently pending, which claims priority to Great Britain Patent Application 0402100.2, filed on Jan. 30, 2004, both of which are hereby incorporated by reference herein.

### BACKGROUND OF THE INVENTION

The present invention relates to the use of certain imidazolylalkyl-pyridines in the treatment of ocular disorders. More particularly, the present invention relates to the use of compounds of formula I, 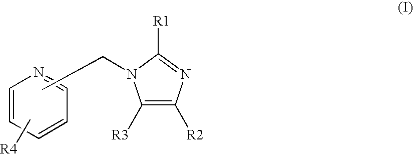
wherein R₁. is hydrogen, lower alkyl, halogen with an atomic number of 9 to 35 or amino optionally mono- or disubstituted by lower alkyl, R₂. and R₃. independently of one another are hydrogen or lower alkyl, R₄. is hydrogen, hydroxy, lower alkyl, lower alkoxy or halogen with an atomic number of 9 to 35, in free base or acid addition salt form, and the bridge between the pyridine and the imidazole, illustrated as methylene, is methylene or ethylene.

### SUMMARY OF THE INVENTION

The present invention provides methods and uses of imidazolylalkyl-pyridines in the treatment of ocular disorders. A first aspect of the invention provides a method of treating ocular neovascularization comprising administering to a subject in need of such treatment a therapeutically effective amount of a compound having the formula (I) in free base or physiologically acceptable acid addition salt form, 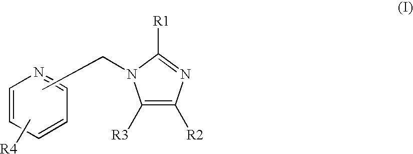
wherein R₁. is hydrogen, lower alkyl, halogen with an atomic number of 9 to 35 or amino optionally mono- or disubstituted by lower alkyl, R₂. and R₃. independently of one another are hydrogen or lower alkyl, R₄. is hydrogen, hydroxy, lower alkyl, lower alkoxy or halogen with an atomic number of 9 to 35, and the bridge between the pyridine and the imidazole, illustrated as methylene, is methylene or ethylene.

A second aspect of the invention provides a use of a compound of formula (I) in the manufacture of a medicament for the treatment of ocular neovascularization, said compound of formula (I) being 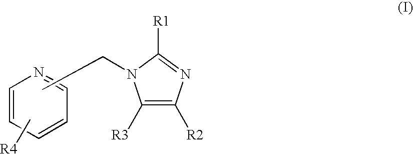
wherein R₁. is hydrogen, lower alkyl, halogen with an atomic number of 9 to 35 or amino optionally mono- or disubstituted by lower alkyl, R₂. and R₃. independently of one another are hydrogen or lower alkyl, R₄. is hydrogen, hydroxy, lower alkyl, lower alkoxy or halogen with an atomic number of 9 to 35, and the bridge between the pyridine and the imidazole, illustrated as methylene, is methylene or ethylene.

### BRIEF DESCRIPTION OF THE DRAWINGS

FIG. 1 illustrates the effect of a compound of the present invention on capillary network formation in cultured human umbilical vein endothelial cells (HUVECs).

### DETAILD DESCRIPTION OF THE INVENTION

As used herein lower in the context with alkyl and alkoxy groups denote a radical having up to 7 carbon atoms, preferably up to 4 carbon atoms and more preferably up to 2 carbon atoms. Consequently, lower alkyl has especially up to 7 carbon atoms, preferably up to 4 carbon atoms, and in particular up to 2 carbon atoms and is, for example, methyl, ethyl, propyl, isopropyl, butyl, tert-butyl, pentyl, or hexyl. Accordingly, lower alkoxy has up to 7 carbon atoms, preferably up to 4 carbon atoms, and in particular up to 2 carbon atoms and is, for example, methoxy, ethoxy, propoxy, butoxy, tert-butoxy or hexyloxy.

Insofar as above-defined lower alkyl or lower alkoxy groups are present in the compounds of formula (I), these preferably have one or two carbon atoms and especially signify methyl or methoxy. The imidazolylmethyl radical is preferably in position 2 of the pyridine. R₁ is preferably methyl or ethyl, more preferably methyl. R₂ and R₃ are preferably each hydrogen. R₄ is preferably methyl, ethyl or hydrogen, more preferably methyl or hydrogen, and in particular hydrogen. The compound A of Example 1 is strongly preferred.

In a particular group of compounds of formula (I), R₁ is lower alkyl, R₂ and R₃ independently of one another are hydrogen or lower alkyl, and R₄ is hydrogen, lower alkyl or halogen with an atomic number of 9 to 35.

In a further particular group of compounds of formula (I), R₁ is methyl, R₂ and R₃ independently of one another are hydrogen or methyl, and R₄ is hydrogen, methyl or halogen with an atomic number of 9 to 35.

Halogen with an atomic number of 9 to 35 denote in particular a fluorine and chlorine residue, more particularly a chlorine residue.

The compounds of formula (I) may be present in free base form or in the form of their acid addition salts, including, for example, hydrogen fumarate and fumarate salt forms. Acid addition salts may be produced from the free bases in known manner, and vice versa.

The compounds of formula (I) are known, e.g., from U.S. Pat. Nos. 5,856,343 and 5,635,521, which are incorporated herein by reference, or may be produced in accordance with known processes, i.e., analogously to known processes.

The compounds of formula I and their physiologically acceptable salts exhibit interesting pharmacological activities in the eye and may therefore be used in accordance thereto. The compounds according to the invention are therefore useful for the treatment of ocular neovascularization.

As used herein the terms “treatment” or “treat” refer to both prophylactic or preventive treatment as well as curative or disease-modifying treatment, including treatment of patients at risk of contracting the disease or suspected to have contracted the disease as well as patients who are ill or have been diagnosed as suffering from a disease or medical condition.

For the indications as described herein, the appropriate dosage will, of course, vary depending upon, for example, the compound employed, the host, the mode of administration and the nature and severity of the condition being treated. However, in general, satisfactory results in animals are indicated to be obtained at daily dosages from about 0.05 to about 50 mg/kg animal body weight. In larger mammals, for example humans, an indicated daily dosage may typically range from about 0.1 mg to about 100 mg, conveniently administered, for example, in divided doses up to four times a day.

The compounds according to the invention may be administered by any conventional route, in particular enterally, orally, or topically, for example in the form of tablets, capsules or eye drops, or parenterally, for example in the form of injectable solutions or suspensions.

The present invention furthermore provides a method of treating ocular neovascularization in a subject in need of such treatment, which comprises administering a therapeutically effective amount of a compound of formula (I). Ocular neovascularization is in particular referring to a medical condition which involves corneal, iris, choroidal, or retinal neovascularization (NV), age related macular degeneration, proliferative diabetic retinopathy, retinopathy of prematurity (ROP), ischemic retinopathy, and central vein occlusion. As used herein age related macular degeneration refers to both the dry and wet form of age related macular degeneration, the wet form being a preferred form. The following examples illustrate the invention.

### EXAMPLE 1

### Compound A

[2-(2-methylimidazol-1-yl)methyl]pyridine (See, e.g., U.S. Pat. No. 5,856,343) 9.7 g (75 μM) of 2-(chloromethyl)pyridine and 42 g (512 μM) of 2- methyl-imidazole are suspended in 40 ml dimethylformamide, then stirred for 3 hours at 105° C. The dimethylformamide is distilled off and the crystalline residue is diluted with ethyl acetate and a little hexane. Following filtration, the mother solution is concentrated by evaporation and the dimethylformamide distilled off, and then shaken out several times between water and methylene chloride. 10.3 g of the oily title compound are obtained.

### Capillary Network Formation in Cultured HUVECs

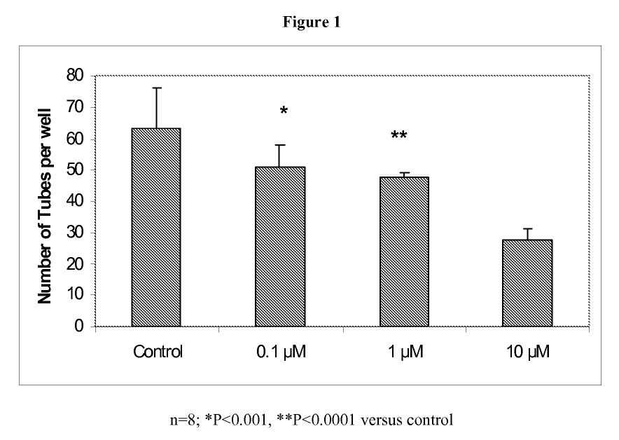

Various concentrations of compound A (0.1, 1, and 10 μM) are tested to assess the quality of effect of this compound on capillary networking (tube formation) in cultured human umbilical vein endothelial cells (HUVECs) seeded on growth factor-reduced Matrigel. The intensity of capillary networking is evaluated at 24 hours using a phase-contrast microscope. Images are captured and analySYS 3.2 software is used to count the number of tubes in five random fields of each well (8 wells altogether). The effect of the compound is compared to untreated cells grown in cell culture medium (control). Compound A significantly decreases the number of tubes at 1 μM and 10 μM concentrations as compared to the control group (FIG. 1). The inhibitory effect of this compound is not related to cell toxicity as this is excluded by an additional study using ToxiLight kit (Cambrex), which is a very sensitive assay based on ATP release from damaged cells.

FIG. 1 illustrates the effect of compound A on capillary network formation in cultured HUVECS. In this figure the bars represent mean ±SD of the number of tubes per group; wells per group, n=8; *P<0.001, **P<0.0001 versus control.

Based on the above finding, compound A and related compounds are considered useful in the treatment of various ocular diseases that result from pathological angiogenesis, also called neovascularization (NV).

1. A method of treating ocular neovascularization comprising administering to a subject in need of such treatment a therapeutically effective amount of a compound having the formula (I) in free base or physiologically acceptable acid addition salt form, 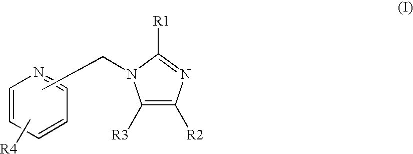
wherein R₁. is hydrogen, lower alkyl, halogen with an atomic number of 9 to 35 or amino optionally mono- or disubstituted by lower alkyl;
R₂. and R₃. independently of one another are hydrogen or lower alkyl;
R₄. is hydrogen, hydroxy, lower alkyl, lower alkoxy or halogen with an atomic number of 9 to 35; and
the bridge between the pyridine and the imidazole, illustrated as methylene, is methylene or ethylene.

2. The method of claim 1 wherein R₁ is lower alkyl, halogen with an atomic number of 9 to 35 or amino optionally mono- or disubstituted by lower alkyl and the bridge between the pyridine and the imidazole is methylene.

3. The method of claim 2 wherein the compound administered is the compound of Formula (I) wherein R₁ is methyl.

4. The method of claim 1 wherein the compound administered is in free base, hydrogen fumarate, or fumarate salt form.

5. The method of claim 1 wherein the compound administered is [2-(2-methylimidazol-1-yl-)methyl]pyridine, in free base or physiologically acceptable acid addition salt form.

6. The method of claim 5 wherein the compound administered is [2-(2-methylimidazol-1-yl)methyl]pyridine in free base, hydrogen fumarate, or fumarate salt form.

7. The method of claim 5 wherein the compound administered is [2-(2-methylimidazol-1-yl)methyl]pyridine fumarate.

8. The method of claim 1, wherein said ocular neovascularization is at least one of choroidal and retinal neovascularization.

9. The method of claim 1, wherein said ocular neovascularization is age related macular degeneration.

10. The method of claim 1, wherein said ocular neovascularization is proliferative diabetic retinopathy.

11. The method of claim 1, wherein said ocular neovascularization is ischemic retinopathy.

12. The method of claim 1 wherein said method is for treating a human or a mammal.

13. A medicament for the treatment of ocular neovascularization comprising a compound of Formula (I) 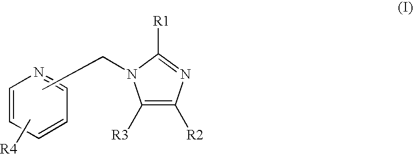
wherein R₁. is hydrogen, lower alkyl, halogen with an atomic number of 9 to 35 or amino optionally mono- or disubstituted by lower alkyl;
R₂. and R₃. independently of one another are hydrogen or lower alkyl;
R₄. is hydrogen, hydroxy, lower alkyl, lower alkoxy or halogen with an atomic number of 9 to 35; and
the bridge between the pyridine and the imidazole, illustrated as methylene, is methylene or ethylene.

14. The medicament of claim 13, wherein said treatment is both prophylactic or preventive treatment as well as curative or disease-modifying treatment, including treatment of patients at risk of contracting the disease or suspected to have contracted the disease as well as patients who are ill or have been diagnosed as suffering from a disease or medical condition.

15. The method of claim 1, wherein said treatment is both prophylactic or preventive treatment as well as curative or disease-modifying treatment, including treatment of patients at risk of contracting the disease or suspected to have contracted the disease as well as patients who are ill or have been diagnosed as suffering from a disease or medical condition.
